(19) United States
(12) Patent Application Publication
(10) Pub. No.: US 2026/0123850 A1
(43) Pub. Date: May 7, 2026

(54) ASSESSMENT OF LUNG CAPACITY, RESPIRATORY FUNCTION, ABDOMINAL STRENGTH AND/OR THORACIC STRENGTH OR IMPAIRMENT

(71) Applicant: Hoffmann-La Roche Inc., Little Falls, NJ (US)

(72) Inventors: Doris BERCHTOLD, Basel (CH); Foteini ORFANIOTOU, Riehen (CH); Thanneer Malai PERUMAL, Oberwil, Basel-Landschaft (CH); Anja Kaja RIES, Basel (CH)

(21) Appl. No.: 19/118,344

(22) PCT Filed: Oct. 6, 2023
(86) PCT No.: PCT/EP2023/077725

§ 371 Date: Apr. 3, 2025

(30) Foreign Application Priority Data
Oct. 7, 2022 (EP) 22200392.3

(51) Int. Cl. A61B 5/08; A61B 5/00; G10L 25/66; G10L 25/78

(52) U.S. Cl. CPC A61B 5/0803; A61B 5/4519; A61B 5/4803; A61B 5/4842; A61B 5/6898; A61B 5/7264; A61B 5/7475; G10L 25/66; G10L 25/78; A61B 2562/0204

(57) ABSTRACT

A diagnostic device configured to assess respiratory function, lung capacity, abdominal strength and/or thoracic strength or impairments of a subject, the diagnostic device comprising: a processor; a microphone; and a memory storing computer-readable instructions that, when executed by the processor, cause the diagnostic device to: prompt the subject to perform a diagnostic task of making a long “aaah” sound for a predetermined duration; receive audio data associated with the diagnostic task via the microphone; extract, from the audio data, digital biomarker data; and apply an analytical model to the extracted digital biomarker data, the analytical model configured to generate an output indicative of the respiratory function, lung capacity, abdominal strength and/or thoracic strength or impairments of the subject

### TECHNICAL FIELD OF THE INVENTION

The present invention relates to diagnostic device and computer-implemented methods configured to assess respiratory function, lung capacity, abdominal strength and/or thoracic strength or impairments of a subject.

### BACKGROUND TO THE INVENTION

People living with spinal muscular atrophy (SMA) report difficulty speaking loudly (e.g. to make themselves heard in a noisy environment), and may experience shortness of breath while speaking.

Moreover, the Scientific Advisory Working Group (SAWG) recommended that combining measurements from speech and respiration assessments could help detect worsening of bulbar function that might foreshadow critical events (such as aspirations). In addition, since people with spinal muscular atrophy report difficulty speaking loudly, it is hypothesized that the sound pressure level¹ of speech might be a further outcome measure. ^{I} This is often incorrectly referred to as “loudness”—loudness is a psychoacoustic term that refers to the subjective perception of sound pressure, and is affected by factors such the frequency-dependent sensitivity of human hearing, and masking effects that are used in audio compression schemes such as MP3. Unless these effects of human hearing are being modeled, the term level should be used.

It is desirable to measure the respiratory function, lung capacity, and abdominal/thoracic/strength/impairment, since this can help to track the status or progression of various conditions, such as SMA. The present inventors have devised a scheme to do so.

### SUMMARY OF THE INVENTION

The present invention provides a diagnostic device and computer-implemented methods of assessing respiratory function, lung capacity, abdominal strength and/or thoracic strength or impairments of a subject of a subject. The outputs may be useful in assessing bulbar function of a subject, and to track the status or progression of conditions affecting bulbar function, such as (but not exclusively) SMA.

More specifically, a first aspect of the present invention provides a diagnostic device configured to assess respiratory function, lung capacity, abdominal strength and/or thoracic strength or impairments of a subject, the diagnostic device comprising: a processor; a microphone; and a memory storing computer-readable instructions that, when executed by the processor, cause the diagnostic device to: prompt the subject to perform a diagnostic task of making a long “aaah” sound for a predetermined duration; receive audio data associated with the diagnostic task via the microphone; extract, from the audio data, digital biomarker data; and apply an analytical model to the extracted digital biomarker data, the analytical model configured to generate an output indicative of the respiratory function, lung capacity, abdominal strength and/or thoracic strength or impairments of the subject.

By measuring respiratory function using a diagnostic device according to the first aspect of the present invention, it may be possible to track, effectively, the progress of various muscular disabilities such as SMA in a subject by active testing of the subject. In particular, the computer-readable instructions, when executed by the processor, may be further configured to cause the diagnostic device to map the output a bulbar function assessment grade indicative of the bulbar function of the subject. As is described in detail later in this application, the diagnostic device according to the first aspect of the present invention may use the output indicative of the respiratory function and/or the bulbar function assessment grade to indicate and/or track the presence or progression of a muscular disability, such as SMA, in a subject or user.

In preferred implementations, the device is or comprises a smartphone. This is advantageous because smartphones are possessed by virtually everyone nowadays. By implementing a computer-implemented process such as the one described on a smartphone, a user need not attend e.g. a hospital or other clinical setting in order for the respiratory function, lung capacity, abdominal strength and/or thoracic strength or impairments of a subject to be measured. Other kinds of diagnostic device may be used, e.g. a tablet, a laptop computer, a desktop computer, or the like. Alternatively, the diagnostic device may be a dedicated diagnostic device for assessing respiratory function, lung capacity, abdominal strength and/or thoracic strength or impairments of a subject.

It is generally preferable to extract the digital biomarker data only from portions of the recorded audio data in which the user is actually vocalizing. However, the recorded audio data may include e.g. background noise before the subject begins performing the diagnostic task, and after they have completed it. More specifically, the audio data may comprise a plurality of segments, and extracting the digital biomarker data may comprise applying a first algorithm to the audio data, the first algorithm configured to classify the segments of the audio data into active speech segments and background noise segments. Herein, an “active speech segment” refers to a segment in which the user is actually performing the diagnostic task. Classifying the segments of the audio data into active speech segments and background noise segments comprises generating timestamps indicating the beginning and end times of each respective active speech segment and background noise segment. The length of the diagnostic task may be 10 to 60 seconds, 15 to 45 seconds, 20 to 40 seconds, or preferably about 30 seconds.

During each active speech segment, there may be times when the subject is making the “aaah” sound, and times when the user has to pause e.g. for breath, to begin another “aaah” sound. These may be referred to voiced speech sub-segments, and non-voiced speech sub-segments respectively. More specifically, each active speech segment may comprise a plurality of sub-segments; and extracting the digital biomarker data may comprise applying a second algorithm to the active speech segments of the audio data, the second algorithm configured to classify the sub-segments into voiced speech sub-segments and non-voiced speech sub-segments. Classification of the sub-segments may be achieved in the same manner as classification of the segments, i.e. classifying the sub-segments of the active speech segments of the audio data into voiced speech segments and non-voiced speech segments may comprise generating timestamps indicating the beginning and end times of each respective voiced speech sub-segment and non-voiced speech sub-segment. The term “voiced speech sub-segment” may correspond to a sub-segment during which the subject's vocal cords or folds are actually vibrating.

We now discuss the nature of the digital biomarker data in more detail, and its extraction. Various types of digital biomarker data may be extracted from the recorded audio data, and the list of examples set out below is by no means exhaustive. Essentially, the types of digital biomarker parameterize various aspects of a subject's respiratory function, lung capacity, abdominal strength and/or thoracic strength, which may be affected by declining bulbar muscular function, e.g. as a result of SMA.

In some cases, the digital biomarker data may comprise a total duration of voiced speech sub-segments within the predetermined duration of the diagnostic task. In these cases, extracting the digital biomarker data may comprise calculating the total duration of voiced speech sub-segments based on e.g. the generated timestamps.

In some cases, the digital biomarker data may comprise a total number of voiced speech sub-segments in the active speech segments of the audio data. In these cases, extracting the digital biomarker data may comprise counting the total duration of voiced speech sub-segments in the active speech segments, based on e.g. the generated timestamps.

In some cases, the digital biomarker data may comprise a total duration of non-voiced speech sub-segments within the predetermined duration of the diagnostic task. In these cases, extracting the digital biomarker data may comprise calculating the total duration of non-voiced speech sub-segments based on e.g. the generated timestamps.

In some cases, the digital biomarker data may comprise one or more of the duration of the longest non-voiced speech sub-segment and the shortest non-voiced speech sub-segment in the active speech segments of the audio data.

When obtaining data such as this, the relative distance and orientation of the microphone relative to the subject's mouth is important, for example to ensure consistency of measurements. Accordingly, in some cases, the computer-readable instructions, when executed by the processor, may further cause the device to prompt the subject to place the device at a pre-determined distance from the subject. Alternatively, or additionally, the computer-readable instructions, when executed by the processor, may further cause the device to prompt the subject to place the device in a pre-determined position.

In some cases the computer-readable instructions, when executed by the processor, may further cause the device to: receive, via the microphone, noise data; calculate, from the noise data, a background noise; and use the background noise to apply a correction to the audio data.

In some examples, the output indicative of the respiratory function of the subject may correspond to the digital biomarker data. For example, the output indicative of the respiratory function may correspond to the total duration of voiced speech sub-segments within the predetermined duration, the total number of voiced speech sub-segments in the active speech segments, the total duration of non-voiced speech sub-segments within the predetermined duration, the duration of the longest non-voiced speech sub-segment in the active speech segments, or the duration of the shortest non-voiced speech sub-segment in the active speech segments.

We now discuss how the output indicative of the respiratory function may be used to indicate a presence or a progression of a muscular disability, such as SMA. The computer-readable instructions, when executed by the at least one processor, may cause the diagnostic device to apply a clinical interpretation model to the output indicative of the respiratory function.

The clinical interpretation model may be configured to output an indication of the presence or absence of a muscular disability, such as SMA, in the user, or an indication of the progression of a muscular disability in the user. The clinical interpretation model may be configured to compare the output indicative of the respiratory function to a predetermined value, and, based on the comparison, to output an indication of the presence or absence of the muscular disability, such as SMA. In particular, the clinical interpretation model may be configured to determine whether the output indicative of the respiratory function is greater than a predetermined threshold. In some examples, the clinical interpretation model may be configured to, if it is determined that the output indicative of the respiratory function is greater than the predetermined threshold, to output an indication of the presence of a muscular disability (e.g., that the user is a PlwSMA), and/or if it is determined that the output indicative of the respiratory function is less than or equal to the predetermined threshold, to output an indication of the absence of the muscular disability. In other examples, the clinical interpretation model may be configured to, if it is determined that the output indicative of the respiratory function is less than the predetermined threshold, to output an indication of the presence of a muscular disability (e.g., that the user is a PlwSMA), and/or if it is determined that the output indicative of the respiratory function is greater than or equal to the predetermined threshold, to output an indication of the absence of the muscular disability.

A second aspect of the present invention provides a computer-implemented method of assessing respiratory function, lung capacity, abdominal strength and/or thoracic strength or impairments of a subject, the computer-implemented method comprising the steps of: prompting the subject to perform a diagnostic task of making a long “aaah” sound for a predetermined duration; receiving audio data associated with the diagnostic task via the microphone; extracting, from the audio data, digital biomarker data; and applying an analytical model to the extracted digital biomarker data, the analytical model configured to generate an output indicative of the respiratory function, lung capacity, abdominal strength and/or thoracic strength or impairments of the subject. In preferred cases, the computer-implemented method of the second aspect of the invention is executed by a processor of a diagnostic device such as the diagnostic device of the first aspect of the invention. It will be appreciated that the optional features set out above, in respect of the first aspect of the invention, apply equally well to the second aspect of the invention except where context clearly dictates otherwise, or whether such a combination of features is clearly technically incompatible.

A third aspect of the invention provides a computer program comprising instructions which when executed by a processor of a computer (or other suitable data processing device) cause the processor to execute the computer-implemented method of the second aspect of the invention. A further aspect of the invention provides a computer-readable storage medium having stored thereon the computer program of the third aspect of the invention.

The invention includes the combination of the aspects and preferred features described except where such a combination is clearly impermissible or expressly avoided.

### BRIEF DESCRIPTION OF THE DRAWINGS

Embodiments of the present invention will now be described with reference to the accompanying drawings, in which:

FIG. 1 is a diagram of an example environment in which a diagnostic device for assessing the respiratory function, lung capacity, abdominal strength and/or thoracic strength or impairments of a subject is provided.

FIG. 2 is a flow diagram of a computer-implemented method for assessing the respiratory function, lung capacity, abdominal strength and/or thoracic strength or impairments of a subject.

FIG. 3 illustrates one example of a network architecture and data processing device that may be used to implement one or more illustrative aspects described herein.

### DETAILED DESCRIPTION OF THE DRAWINGS

Aspects and embodiments of the present invention will now be discussed with reference to the accompanying figures. Further aspects and embodiments will be apparent to those skilled in the art. All documents mentioned in this text are incorporated herein by reference.

In the following description of various aspects, reference is made to the accompanying drawings, which form a part hereof, and in which is shown by way of illustration various embodiments in which aspects described herein may be practiced. It is to be understood that other aspects and/or embodiments may be utilized, and structural and functional modifications may be made without departing from the scope of the described aspects and embodiments.

Aspects described herein are capable of other embodiments and of being practiced or being carried out in various ways. Also, it is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed Thereafter and equivalents thereof as well as additional items and equivalents thereof. The use of the terms “mounted,” “connected,” “coupled,” “positioned,” “engaged” and similar terms, is meant to include both direct and indirect mounting, connecting, coupling, positioning and engaging.

Systems, methods and devices described herein provide a diagnostic device and computer-implemented methods for assessing, measuring, or determining the respiratory function, lung capacity, abdominal strength and/or thoracic strength or impairments of a subject, for example a patient suffering from a muscular disability, such as particular SMA. In some cases, the diagnostic device may be in the form of a mobile, in particular a smartphone, on which a particular software application is installed. The software application may be configured to execute (or cause the processor of the mobile device) the corresponding computer-implemented method.

In some cases, the diagnostic obtains or receives sensor data from one or more sensors associated with the mobile device as the subject interacts with the software application using the mobile device. In some cases, the sensors may be within the mobile device. In some cases, the data indicative of respiratory function, lung capacity, abdominal strength and/or thoracic strength or impairments of a subject is derived, calculated, or extracted from the received or obtained sensor data. In some cases, the assessment of the symptom severity and progression of a muscular disability, in particular SMA, in the subject may be determined based on the extracted sensor features.

In implementations of the present invention, the diagnostic device may prompt the subject to perform a diagnostic tasks. In some cases, the diagnostic tasks are anchored in or modelled after established methods and standardized tests. In some cases, in response to the subject performing the diagnostic task, the diagnostic obtains or receives sensor data via one or more sensors. In some cases, the sensors may be within a mobile device or wearable sensors worn by the subject. In some cases, sensor features associated with the symptoms of a muscular disability, in particular SMA, are extracted from the received or obtained sensor data. In some cases, the assessment of the symptom severity and progression of a muscular disability, in particular SMA, in the subject is determined based on the extracted features of the sensor data.

Assessments of symptom severity and progression of a muscular disability, in particular SMA, using diagnostics according to the present disclosure correlate sufficiently with the assessments based on clinical results and may thus replace clinical subject monitoring and testing. Example diagnostics according to the present disclosure may be used in an out of clinic environment, and therefore have advantages in cost, ease of subject monitoring and convenience to the subject. This facilitates frequent, in particular daily, subject monitoring and testing, resulting in a better understanding of the disease stage and provides insights about the disease that are useful to both the clinical and research community. An example diagnostic according to the present disclosure can provide earlier detection of even small changes in respiratory function, lung capacity, abdominal strength and/or thoracic strength or impairments of a subject which can be indicative of the presence or progression of muscular disabilities, in particular SMA, in a subject and can therefore be used for better disease management including individualized therapy.

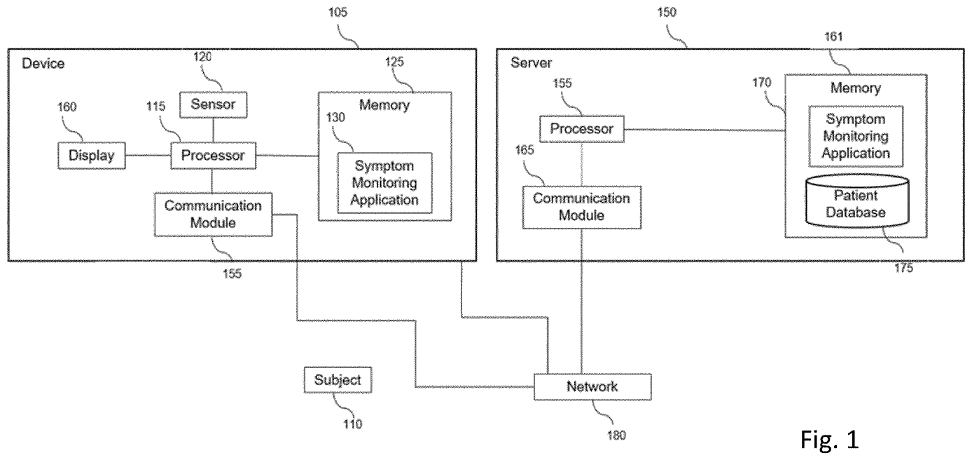

FIG. 1 is a diagram of an example environment in which a diagnostic device 105 for assessing respiratory function, lung capacity, abdominal strength and/or thoracic strength or impairments of a subject 110. In some cases, the device 105 may be a smartphone, a smartwatch or other mobile computing device. The device 105 includes a display screen 160. In some cases, the display screen 160 may be a touchscreen. The device 105 includes at least one processor 115 and a memory 125 storing computer-instructions for a symptom monitoring application 130 that, when executed by the at least one processor 115, cause the device 105 to assess respiratory function, lung capacity, abdominal strength and/or thoracic strength or impairments of a subject. The device 105 receives a plurality of sensor data via one or more sensors associated with the device 105. In some cases, the one or more sensors associated with the device is at least one of a sensor disposed within the device or a sensor worn by the subject and configured to communicate with the device. In FIG. 1, the sensors associated with the device 105 include a first sensor 120 such as a microphone which is located in device 105.

The device 105 extracts, from the received first sensor data, digital biomarker data, which can be used to determine respiratory function, lung capacity, abdominal strength and/or thoracic strength or impairments of a subject.

The device 105 determines the respiratory function, lung capacity, abdominal strength and/or thoracic strength or impairments of a subject 110 based on the extracted features. In some cases, the device 105 sends the extracted features over a network 180 to a server 150. In some cases, the device 105 sends the first sensor data over the network 180 to the server 150. The server 150 includes at least one processor 155 and a memory 161 storing computer-instructions for a symptom assessment application 170 that, when executed by the server processor 155, cause the processor 155 to determine respiratory function, lung capacity, abdominal strength and/or thoracic strength or impairments of a subject 110 based on the extracted features received by the server 150 from the device 105. In some cases, the symptom assessment application 170 may cause the processor 115 to extract the features from the sensor data received from the device 105. In some cases, the symptom assessment application 170 may determine the respiratory function, lung capacity, abdominal strength and/or thoracic strength or impairments of a subject 110 based on the extracted features of the sensor data, which may be received from the device 105, and a subject database 175 stored in the memory 160. In some cases, the subject database 175 may include subject and/or clinical data. In some cases, the subject database 175 may include in-clinic and sensor-based measures of the respiratory function, lung capacity, abdominal strength and/or thoracic strength or impairments. In some cases, the subject database 175 may be independent of the server 150. In some cases, the server 150 sends the determined respiratory function, lung capacity, abdominal strength and/or thoracic strength or impairments of a subject 110 to the device 105. In some cases, the device 105 may output the respiratory function, lung capacity, abdominal strength and/or thoracic strength or impairments of a subject 110. In some cases, the device 105 may communicate information to the subject 110 based on the assessment. In some cases, the assessment of respiratory function, lung capacity, abdominal strength and/or thoracic strength or impairments of a subject 110, may be communicated to a clinician that may determine individualized therapy for the subject 110 based on the assessment.

In some cases, the computer-instructions for the symptom monitoring application 130, when executed by the at least one processor 115, cause the device 105 to determine the respiratory function, lung capacity, abdominal strength and/or thoracic strength or impairments of a subject 110 based on active testing of the subject 110. The device 105 prompts the subject 110 to perform one or more tasks. In some cases, prompting the subject to perform the one or more diagnostic tasks includes prompting the subject to make a continuous “aaah” sound for as long as possible.

In response to the subject 110 performing the one or more diagnostic tasks, the diagnostic device 105 receives a plurality of sensor data via the one or more sensors associated with the device 105. The device 105 extracts, from the received sensor data various digital biomarker data, from which an assessment of respiratory function, lung capacity, abdominal strength and/or thoracic strength or impairments of a subject 110 may be made. The symptoms of a muscular disability, in particular SMA in the subject 110 may include a symptom affecting of the respiratory function, lung capacity, abdominal strength and/or thoracic strength or impairments of a subject 110.

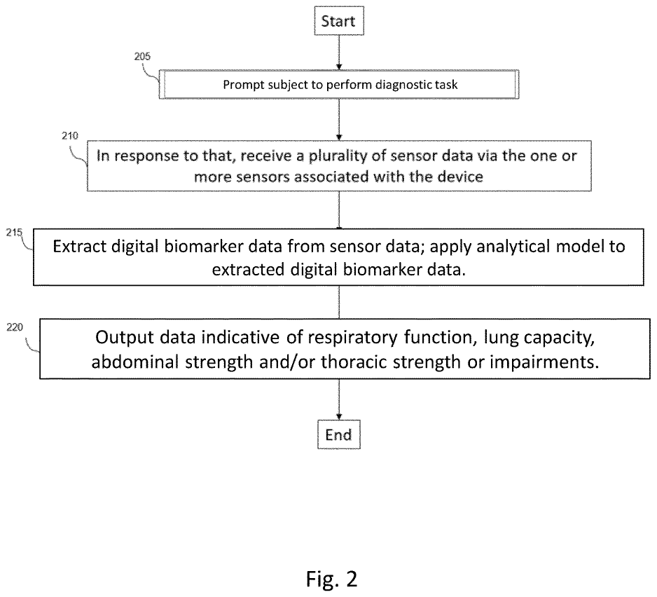

FIG. 2 illustrates an example method for assessing the respiratory function, lung capacity, abdominal strength and/or thoracic strength or impairments of a subject 110 based on active testing of the subject using the example device 105 of FIG. 1. While FIG. 2 is described with reference to FIG. 1, it should be noted that the method steps of FIG. 2 may be executed by other systems. The computer-implemented method includes, in step 205, prompting the subject to perform a diagnostic task as outlined above. The method includes receiving, in response to the subject performing the one or more tasks, a plurality of sensor data, via e.g. a microphone (step 210).

Then, in step 215, digital biomarker data is extracted from the sensor data, and an analytical model is applied to the digital biomarker data.

In step 220, data indicative of a respiratory function, lung capacity, abdominal strength and/or thoracic strength or impairments of a subject is output, e.g. by the processor 107 generating instructions, which when executed by the display component 160 of the device 105 cause the display component 160 to display an output indicative of the respiratory function, lung capacity, abdominal strength and/or thoracic strength or impairments of a subject. Alternatively, the calculated data indicative of the respiratory function, lung capacity, abdominal strength and/or thoracic strength or impairments of a subject may be transmitted to a server 150, as outlined elsewhere in this application.

As discussed above, assessments of symptom severity and progression of a muscular disability, in particular SMA using diagnostics according to the present disclosure correlate sufficiently with the assessments based on clinical results and may thus replace clinical subject monitoring and testing.

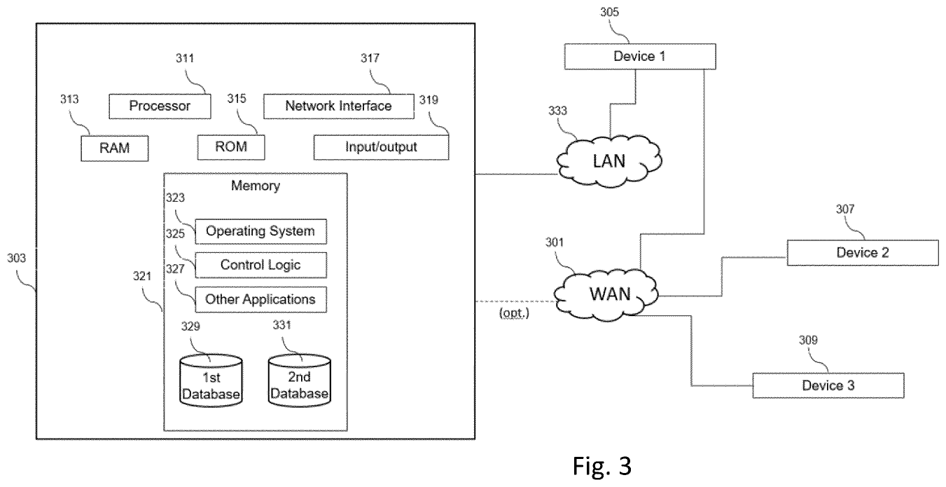

FIG. 3 illustrates an example of a network architecture and data processing device that may be used to implement one or more illustrative aspects described herein, such as the aspects described in FIGS. 1 and 2. Various network nodes 303, 305, 307, and 309 may be interconnected via a wide area network (WAN) 301, such as the Internet. Other networks may also or alternatively be used, including private intranets, corporate networks, LANs, wireless networks, personal networks (PAN), and the like. Network 301 is for illustration purposes and may be replaced with fewer or additional computer networks. A local area network (LAN) may have one or more of any known LAN topology and may use one or more of a variety of different protocols, such as Ethernet. Devices 303, 305, 307, 309 and other devices (not shown) may be connected to one or more of the networks via twisted pair wires, coaxial cable, fibre optics, radio waves or other communication media.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data-attributable to a single entity which resides across all physical networks.

The components may include data server 303, web server 305, and client computers 307, 309. Data server 303 provides overall access, control and administration of databases and control software for performing one or more illustrative aspects described herein. Data server 303 may be connected to web server 305 through which users interact with and obtain data as requested. Alternatively, data server 303 may act as a web server itself and be directly connected to the Internet. Data server 303 may be connected to web server 305 through the network 301 (e.g., the Internet), via direct or indirect connection, or via some other network. Users may interact with the data server 303 using remote computers 307, 309, e.g., using a web browser to connect to the data server 303 via one or more externally exposed web sites hosted by web server 305. Client computers 307, 309 may be used in concert with data server 303 to access data stored therein, or may be used for other purposes. For example, from client device 307 a user may access web server 305 using an Internet browser, as is known in the art, or by executing a software application that communicates with web server 305 and/or data server 303 over a computer network (such as the Internet). In some cases, the client computer 307 may be a smartphone, smartwatch or other mobile computing device, and may implement a diagnostic device, such as the device 105 shown in FIG. 1. In some cases, the data server 303 may implement a server, such as the server 150 shown in FIG. 1.

Servers and applications may be combined on the same physical machines, and retain separate virtual or logical addresses, or may reside on separate physical machines. FIG. 1 illustrates just one example of a network architecture that may be used, and those of skill in the art will appreciate that the specific network architecture and data processing devices used may vary, and are secondary to the functionality that they provide, as further described herein. For example, services provided by web server 305 and data server 303 may be combined on a single server.

Each component 303, 305, 307, 309 may be any type of known computer, server, or data processing device. Data server 303, e.g., may include a processor 311 controlling overall operation of the rate server 303. Data server 303 may further include RAM 313, ROM 315, network interface 317, input/output interfaces 319 (e.g., keyboard, mouse, display, printer, etc.), and memory 321. I/O 319 may include a variety of interface units and drives for reading, writing, displaying, and/or printing data or files. Memory 321 may further store operating system software 323 for controlling overall operation of the data processing device 303, control logic 325 for instructing data server 303 to perform aspects described herein, and other application software 327 providing secondary, support, and/or other functionality which may or may not be used in conjunction with other aspects described herein. The control logic may also be referred to herein as the data server software 325. Functionality of the data server software may refer to operations or decisions made automatically based on rules coded into the control logic, made manually by a user providing input into the system, and/or a combination of automatic processing based on user input (e.g., queries, data updates, etc.).

Memory 321 may also store data used in performance of one or more aspects described herein, including a first database 329 and a second database 331. In some cases, the first database may include the second database (e.g., as a separate table, report, etc.). That is, the information can be stored in a single database, or separated into different logical, virtual, or physical databases, depending on system design. Devices 305, 307, 309 may have similar or different architecture as described with respect to device 303. Those of skill in the art will appreciate that the functionality of data processing device 303 (or device 305, 307, 309) as described herein may be spread across multiple data processing devices, for example, to distribute processing load across multiple computers, to segregate transactions based on geographic location, user access level, quality of service (Qos), etc.

One or more aspects described herein may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer-readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects, and such data structures are contemplated within the scope of computer executable instructions and computer-usable data described herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A diagnostic device configured to assess respiratory function, lung capacity, abdominal strength and/or thoracic strength or impairments of a subject, the diagnostic device comprising:
a processor;
a microphone; and
a memory storing computer-readable instructions that, when executed by the processor, cause the diagnostic device to:
prompt the subject to perform a diagnostic task of making a long “aaah” sound for a predetermined duration;
receive audio data associated with the diagnostic task via the microphone;
extract, from the audio data, digital biomarker data; and
apply an analytical model to the extracted digital biomarker data, the analytical model configured to generate an output indicative of the respiratory function, lung capacity, abdominal strength and/or thoracic strength or impairments of the subject.

2. A diagnostic device according to claim 1, wherein:
the audio data comprises a plurality of segments; and
extracting the digital biomarker data comprises applying a first algorithm to the audio data, the first algorithm configured to classify the segments of the audio data into active speech segments and background noise segments.

3. A diagnostic device according to claim 2, wherein:
classifying the segments of the audio data into active speech segments and background noise segments comprises generating timestamps indicating the beginning and end times of each respective active speech segment and background noise segment.

4. A diagnostic device according to claim 2 wherein:
each active speech segment comprises a plurality of sub-segments; and
extracting the digital biomarker data comprises applying a second algorithm to the active speech segments of the audio data, the second algorithm configured to classify the sub-segments into voiced speech sub-segments and non-voiced speech sub-segments.

5. A diagnostic device according to claim 4, wherein:
classifying the sub-segments of the active speech segments of the audio data into voiced speech segments and non-voiced speech segments comprises generating timestamps indicating the beginning and end times of each respective voiced speech sub-segment and non-voiced speech sub-segment.

6. A diagnostic device according to claim 5, wherein:
the digital biomarker data comprises a total duration of voiced speech sub-segments within the predetermined duration of the diagnostic task.

7. A diagnostic device according to claim 5 wherein:
the digital biomarker data comprises a total number of voiced speech sub-segments in the active speech segments of the audio data.

8. A diagnostic device according to claim 5, wherein:
the digital biomarker data comprises one or more of the duration of the longest voice speech sub-segment and the shortest voiced speech sub-segment in the active speech segments of the audio data.

9. A diagnostic device according to claim 5, wherein:
the digital biomarker data comprises a total duration of non-voiced speech sub-segments within the predetermined duration of the diagnostic task.

10. A diagnostic device according to claim 5, wherein:
the digital biomarker data comprises one or more of the duration of the longest non-voiced speech sub-segment and the shortest non-voiced speech sub-segment in the active speech segments of the audio data.

11. A diagnostic device according to claim 1, wherein:
the computer-readable instructions, when executed by the processor, further cause the device to prompt the subject to place the device at a pre-determined distance from the subject.

12. A diagnostic device according to claim 1, wherein:
the computer-readable instructions, when executed by the processor, further cause the device to prompt the subject to place the device in a pre-determined position.

13. A diagnostic device according to claim 1, wherein:
the computer-readable instructions, when executed by the processor, further cause the device to:
receive, via the microphone, noise data;
calculate, from the noise data, a background noise; and
use the background noise to apply a correction to the audio data.

14. A diagnostic device according to claim 1, wherein:
the audio data is received over a period of 30 seconds.

15. A diagnostic device according to claim 1, wherein:
the device is a smartphone.

16. A diagnostic device according to claim 1 wherein the computer-readable instructions, when executed by the at least one processor, cause the diagnostic device to apply a clinical interpretation model to the output indicative of the respiratory function, wherein the clinical interpretation model outputs an indication of the presence or absence of a muscular disability.

17. A diagnostic device according to claim 16, wherein the clinical interpretation model is configured to compare the output indicative of the respiratory function to a predetermined value, and, based on the comparison, to output an indication of the presence or absence of the muscular disability.

18. A diagnostic device according to claim 17, wherein the clinical interpretation model is configured to:
determine whether the output indicative of the respiratory function is greater than a predetermined threshold; and,
if it is determined that the output indicative of the respiratory function is greater than the predetermined threshold, to output an indication of the presence of a muscular disability; and,
if it is determined that the output indicative of the respiratory function is less than or equal to the predetermined threshold, to output an indication of the absence of the muscular disability.

19. A diagnostic device according to claim 17, wherein the clinical interpretation model is configured to:
determine whether the output indicative of the respiratory function is less than a predetermined threshold; and,
if it is determined that the output indicative of the respiratory function is less than the predetermined threshold, to output an indication of the presence of a muscular disability; and,
if it is determined that the output indicative of the respiratory function is greater than or equal to the predetermined threshold, to output an indication of the absence of the muscular disability.

20. A computer-implemented method of configured to assess respiratory function, lung capacity, abdominal strength and/or thoracic strength or impairments of a subject, the method comprising:
prompting the subject to perform a diagnostic task of making a long “aaah” sound for a predetermined duration;
receiving audio data associated with the diagnostic task via the microphone;
extracting, from the audio data, digital biomarker data; and
applying an analytical model to the extracted digital biomarker data, the analytical model configured to generate an output indicative of the respiratory function, lung capacity, abdominal strength and/or thoracic strength or impairments of the subject.

21. A computer-implemented method according to claim 20, wherein the computer-implemented method further comprises the steps of:
applying a clinical interpretation model to the output indicative of the respiratory function, wherein the clinical interpretation model outputs an indication of the presence or absence of a muscular disability, or an indication of the progression of a muscular disability

22. (canceled)

23. A computer-implemented method according to claim 20, wherein the steps of prompting the subject and receiving the audio data are carried out by a processor of a diagnostic device, and wherein the steps of extracting the digital biomarker data and applying the respiratory function assessment model are carried out by a processor of a server, wherein the diagnostic device is configured to transmit the audio data to the server, and wherein the diagnostic device comprises:
at least one processor;
a microphone; and
a memory storing computer-readable instructions that, when executed by the at least one processor, cause the diagnostic device to:
prompt the subject to perform a diagnostic task of making a long “aaah” sound for a predetermined duration;
receive, via the microphone, audio data associated with the diagnostic task.
